(19) United States
(12) Patent Application Publication
(10) Pub. No.: US 2023/0120340 A1
(43) Pub. Date: Apr. 20, 2023

(54) OCULAR TREATMENT COMPOSITIONS AND METHODS

(71) Applicant: The Johns Hopkins University, Baltimore, MD (US)

(72) Inventors: Jennifer H. Elisseeff, Baltimore, MD (US); Liam Chung, Baltimore, MD (US); Xiaokun Wang, Baltimore, MD (US)

(21) Appl. No.: 18/068,598

(22) Filed: Dec. 20, 2022

Related U.S. Application Data
(63) Continuation of application No. PCT/US2021/039565, filed on Jun. 29, 2021.
(60) Provisional application No. 63/047,219, filed on Jul. 1, 2020.

(51) Int. Cl. A61K 9/16; A61P 37/04; A61K 9/00; A61P 27/02; A61L 27/36

(52) U.S. Cl. CPC A61K 9/16; A61K 9/0048; A61L 27/3633; A61P 27/02; A61P 37/04; A61L 2430/16

(57) ABSTRACT

Provided herein, inter alia, are methods of treating an ocular injury, disease or disorder with use of one or more extracellular matrix materials.

### CROSS REFERENCE TO RELATED APPLICATION

This application claims priority to U.S. Provisional Application No. 63/047,219, filed Jul. 1, 2020, which is hereby incorporated by reference in its entirety.

### STATEMENT OF GOVERNMENT INTERESTS

This invention was made with government support under grant R01EY029055 awarded by the National Institutes of Health. The government has certain rights in the invention.

### FIELD

Embodiments of the invention are related to tissue matrix particles and methods of using the same. The methods include reducing corneal scarring by inducing an immune response.

### BACKGROUND

The cornea serves as a window to the eye, and transparency is central to its function. When the cornea structure is damaged, fibrotic scar tissue can develop and cause corneal opacity and obstructs vision.

There is a significant need for efficacious therapeutic options to treat traumatic injuries from physical and chemical abrasions, as well as cornea damage associated with the rising number of elective cornea surgical procedures.

### SUMMARY

We now provide new compositions and methods that are particularly useful for corneal healing, repair and/or regeneration as well as to treat ocular inflammatory diseases and disorders.

In preferred aspects, the present treatment compositions can stimulate corneal tissue renewal, including through providing a pre-regenerative environment.

In particular aspects, compositions are provided that comprise a biological scaffold material that upon administration to a subject can exhibit a type 2 immune response.

In additional aspects, compositions are provided that comprise a biological scaffold material that upon administration can provide an increase in IL4 production of treated tissue.

Preferred compositions may suitably comprise biocompatible synthetic material, a biomaterial(s), an extracellular matrix material (EM or ECM) or combinations thereof.

In certain embodiments, compositions that comprise extracellular matrix materials are preferred. Preferred extracellular matrix materials, include for example, urinary bladder matrix (UBM).

In particular aspects, preferred compositions will be in a gel form, including as a hydrogel. For instance, suitable and preferred gel composition may comprise an enzymatically or chemically digested extracellular matrix material.

In additional embodiments, composition that comprise a plurality of particles may be preferred, including where the particles have a mean size of about 50 microns or less.

In one aspect, a type 2 immune response as referred to herein may be characterized by the differentiation of CD4⁺ T helper type 2 (Th2) cells and/or the production of one or more of type 2 cytokines interleukin-4 (IL-4), IL-5, IL-9 and/or IL-13, particularly increased production of IL-4 relative to a control (e.g. untreated tissue).

Thus, suitable and preferred compositions, including for use in the present methods of treatment, may be identified empirically for a type 2 immune response. Exemplary protocols are disclosed in the examples which follow and include assessment of an increase IL4 and/or T_{H}2 cells. In certain aspects, a composition providing a type 2 immune response as referred to herein is assessed by a measured increase of IL4 in corneal tissue following administration of the composition by a protocol of Example 2 which follows, including increased production of IL-4 relative to a control (e.g. untreated tissue) such as a 5, 10, 20, 30, 40 50% or more increase in IL4 relative to a control.

Similarly, suitable and preferred compositions, including for use in the present methods of treatment, also may be identified empirically for increased IL4 production following administration of a composition to a subject. In certain aspects, a composition providing an increase of IL4 production to herein is assessed by a measured increase of IL4 in corneal tissue following administration of the composition by a protocol of Example 2 which follows, including increased production of IL-4 relative to a control (e.g. untreated tissue) such as a 5, 10, 20, 30, 40 50% or more increase in IL4 relative to a control.

As referred to herein, a type 2 immune agonist composition will exhibit a type 2 immune response and/or increased IL4 production as referred to herein..

In certain embodiments, the biocompatible scaffold further comprises one or more immune cell modulating agents and/or cells. The immune cell modulating agents suitably may comprise for example cytokines, monokines, chemokines, checkpoint agents, adjuvants, vaccines, antigens, therapeutic agents or combinations thereof.

In another aspect, methods are provided to provide healing, repair or regeneration of corneal tissue, the methods comprising administering to a subject such as a human in need thereof a biocompatible scaffold. Preferably an administered composition that comprises the biocompatible scaffold material can function as a type 2 immune agonist and/or provide increased levels of IL4. In preferred aspects, the scaffold material comprises a plurality of particles comprising one or more of biocompatible synthetic material, a biomaterial(s), an extracellular matrix or combinations thereof. In particular aspects, the particles have a mean size of about 50 microns or less.

In a further aspect, methods are provided to treat an ocular inflammatory disease or disorder, the methods comprising administering to a subject such as a human in need thereof a biocompatible scaffold material. Preferably, an administered composition comprising the biocompatible scaffold material can function as a type 2 immune agonist and/or provide increased levels of IL4.. In preferred aspects, the scaffold material comprises a plurality of particles that comprises one or more of biocompatible synthetic material, a biomaterial(s), an extracellular matrix or combinations thereof. In particular aspects, the particles have a mean size of about 50 microns or less.

In particular aspects, methods are provided to treat uveitis, severe conjunctivitis (vernal keratoconjunctivitis), dry eye syndrome (including, but not limited to, Keratoconjunctivitis sicca and Sjogren’s syndrome), diabetic retinopathy, or autoimmune ocular inflammatory disease.

In particular aspects, a subject will be identified and selected for treatment as disclosed herein, such as to provide healing, repair or regeneration of corneal tissue, or to treat an ocular inflammatory disease or disorder, and then a therapeutic composition will be administered to the identified and selected subject. For instance, a patient may be identified and selected as having suffered physical ocular trauma and in need of corneal healing and that identified patient may be administered a biocompatible scaffold that comprises extracellular matrix material(s) such as urinary bladder matrix (UBM) as disclosed herein to thereby provide corneal healing.

In a further aspect, pharmaceutical compositions are provided comprising a Type 2 immune agonist (e.g. biocompatible scaffold that comprises extracellular matrix material(s) such as urinary bladder matrix (UBM)) as disclosed herein. The compositions suitably may comprise one or more pharmaceutically acceptable carriers. In preferred embodiments, the compositions may be formulated or otherwise adapted for treatment of a disease or disorder as disclosed herein. In preferred aspects, the composition may be formulated as a fluid composition, including as an eye drop composition.

In a yet further aspect, kits are provided for use to for treatment as disclosed herein, such as to provide healing, repair or regeneration of corneal tissue, or to treat an ocular inflammatory disease or disorder. Kits of the invention suitably may comprise 1) one or more biocompatible scaffold materials that suitably comprises extracellular matrix material(s) such as urinary bladder matrix (UBM); and 2) instructions for using the one or more biocompatible scaffold materials. Preferably, a kit will comprise a therapeutically effective amount of one or more biocompatible scaffold materials. The instructions suitably may be in written form, including as a product label.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value or range. Alternatively, particularly with respect to biological systems or processes, the term can mean within an order of magnitude within 5-fold, and also within 2-fold, of a value. Where particular values are described in the application and claims, unless otherwise stated the term “about” meaning within an acceptable error range for the particular value should be assumed.

As used herein, the term “agent” is meant to encompass any molecule, chemical entity, composition, drug, therapeutic agent, chemotherapeutic agent, or biological agent capable of preventing, ameliorating, or treating a disease or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

By “ameliorate” is meant decrease, suppress, attenuate, diminish, arrest, or stabilize the development or progression of a disease.

The term “combination therapy”, as used herein, refers to those situations in which two or more different pharmaceutical agents are administered in overlapping regimens so that the subject is simultaneously exposed to both agents. When used in combination therapy, two or more different agents may be administered simultaneously or separately. This administration in combination can include simultaneous administration of the two or more agents in the same dosage form, simultaneous administration in separate dosage forms, and separate administration. That is, two or more agents can be formulated together in the same dosage form and administered simultaneously. Alternatively, two or more agents can be simultaneously administered, wherein the agents are present in separate formulations. In another alternative, a first agent can be administered just followed by one or more additional agents. In the separate administration protocol, two or more agents may be administered a few minutes apart, or a few hours apart, or a few days apart.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—-or, as appropriate, equivalents thereof—-and that other elements can be included and still fall within the scope/definition of the defined item, composition, apparatus, method, process, system, etc.

As used herein, the term “cytokine” refers generically to proteins released by one cell population that act on another cell as intercellular mediators or have an autocrine effect on the cells producing the proteins. Examples of such cytokines include lymphokines, monokines; interleukins (“ILs”) such as IL-1, IL-1α, IL-2, IL-3, IL-4, IL-5, IL-6, IL-7, IL-8, IL-9, IL-10, IL-11, IL-12, IL-13, IL-15, IL-17A-F, IL-18 to IL-29 (such as IL-23), IL-31, including PROLEUKIN™ rIL-2; a tumor-necrosis factor such as TNF-α or TNF-β, TGF-β1-3; and other polypeptide factors including leukemia inhibitory factor (“LIF”), ciliary neurotrophic factor (“CNTF”), CNTF-like cytokine (“CLC”), cardiotrophin (“CT”), and kit ligand (“KL”).

As used herein, the term “immune cells” generally includes white blood cells (leukocytes) which are derived from hematopoietic stem cells (HSC) produced in the bone marrow “Immune cells” includes, e.g., lymphocytes (T cells, B cells, natural killer (NK) cells) and myeloid-derived cells (neutrophil, eosinophil, basophil, monocyte, macrophage, dendritic cells).

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to the targeted disease or disorder. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

As used herein, “modulating” refers to an increase or decrease in an adaptive immune system response. In a preferred embodiment, this relates to an increased, up-regulated or enhanced adaptive immune system response. An effective amount of an immunomodulatory agent is an amount that when applied or administered in accordance to the techniques herein is sufficient to modulate, preferably up-regulate, an adaptive immune system response.

As used in this specification and the appended claims, the term “or” is generally employed in its sense including “and/or” unless the content clearly dictates otherwise.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, the terms prognostic and predictive information are used interchangeably to refer to any information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient’s disease will respond to a particular therapy (wherein response may be defined in any of a variety of ways). Prognostic and predictive information are included within the broad category of diagnostic information.

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

“Pharmaceutical agent,” also referred to as a “drug,” or “therapeutic agent” is used herein to refer to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (USP), Goodman and Gilman’s The Pharmacological Basis of Therapeutics, 12^{th} Ed., McGraw Hill, 2001; Katzung, B. (ed.) Basic and Clinical Pharmacology, McGraw-Hill/Appleton & Lange; 8^{th} edition (Sep. 21, 2000); Physician’s Desk Reference (Thomson Publishing), and/or The Merck Manual of Diagnosis and Therapy, 18^{th} ed. (2006), or the 19^{th} ed (2011), Robert S. Porter, MD., Editor-in-chief and Justin L. Kaplan, MD., Senior Assistant Editor (eds.), Merck Publishing Group, or, in the case of animals, The Merck Veterinary Manual, 10^{th} ed., Cynthia M. Kahn, B.A., M.A. (ed.), Merck Publishing Group, 2010.

The terms “prevent”, “preventing”, “prevention”, “prophylactic treatment” and the like refer to the administration of an agent or composition to a clinically asymptomatic individual who is at risk of developing, susceptible, or predisposed to a particular adverse condition, disorder, or disease, and thus relates to the prevention of the occurrence of symptoms and/or their underlying cause.

As defined herein, a “therapeutically effective” amount of a compound or agent (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Other aspects are described infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-D show immune profile after cornea trauma. FIG. 1A shows healthy and cornea with abrasion wound at day 14 post-surgery. Tissue haze and damage is identified with arrows. FIG. 1B shows flow cytometry graphs of myeloid cells and T cells in corneas at day 2 and day 7 post surgery. FIG. 1C shows local tissue CD11b+ myeloid cell population change in corneas over time. The monocyte (Ly6c+) and neutrophil (Ly6g+) population increased at day 2 post-surgery, and then reduced by day 7. Macrophages (F480+), and eosinophils (SiglecF+) also increased at day 2 post-surgery and remained at high level. FIG. 1D shows CD3+ T cell populations and T helper CD4+ and CD8+ effector T cells in the cornea. All data is from 6 corneas pooled per group. Control corneas did not receive a wound.

FIGS. 2A-2C show type 2 response enhances corneal wound healing. FIG. 2A shows corneal wounds treated with PBS and Th2 agonists, micro-UBM and nano-UBM. Tissue haze and damage is identified with red circles, followed by immunofluorescence staining of αSMA. FIG. 2B shows SEM images comparing the morphology between micro versus nano-UBM particles. Particle size analysis showing the diameter differences between micro- and nano-UBM particles. FIG. 2C shows corneal scar ratio at day 14 (n=5). FIG. 2D shows quantified expression of IL4 by CD4+ T cells and Eosinophil (CD1 1b+SiglecF+) in draining lymph nodes at day 14 (n=4), *p < 0.05 Vs. PBS, *p < 0.05 Vs. Control.

FIGS. 3A-B show type 2 response alter fibroblast plasticity. FIG. 3A shows flow cytometry plot represents myofibroblast (CD45-CD31-CD29+Thy1.2+αSMA+) in non-treated (PBS) and UBM treated compared to healthy control. FIG. 3B shows quantified expression of CD140a, Sca1, S100a4, αSMA in fibroblasts isolated from the cornea at day 14 (n=4), *p < 0.05 Vs. PBS, *p < 0.05 Vs. Control.

### DETAILED DESCRIPTION

As discussed, in one aspect, we now provide methods of treating an ocular injury, disease or disorder in a subject, comprising administering to subject in need thereof an effective amount of a composition comprising one or more biological scaffold materials, wherein the composition increases IL4 production.

In a further aspect, we provide method of treating an ocular injury, disease or disorder in a subject, comprising administering to subject in need thereof an effective amount of a composition that comprises one or more extracellular matrix materials.

Subjects for treatment include for example a subject that is in need of cornea repair or reconstruction, suh as following injury or surgery. Additional subjects may include those suffering from an inflammatory ocular disease or disorder. Further subject may include those that are suffering from uveitis, severe conjunctivitis (vernal keratoconjunctivitis), and dry eye syndrome (including, but not limited to, Keratoconjunctivitis sicca and Sjogren’s syndrome), diabetic retinopathy, or autoimmune ocular inflammatory disease.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Immune cells rapidly infiltrate into the limbus after a cornea scrape injury model, reaching a peak cell number at 12 hours [4]. Cells continue to infiltrate into the peripheral cornea, maintaining high levels 12 to 36 hours after injury. Central cornea inflammation decreases to a baseline level after 1 week. While macrophages are implicated in causing corneal haze and scarring, macrophage depletion also impairs the corneal wound healing process [5]. Thus, immune cell infiltration and the type of immune response mediates the balance of stroma regeneration and scar formation. After this initial keratocyte apoptosis, the remaining keratocytes activate and undergo proliferation and migrate to the wounded region. The conversion of keratocytes to corneal fibroblasts or myofibroblasts is mediated by transforming growth factor beta (TGF-β) [6]. The over-activation of myofibroblasts in the wound significantly reduces the stromal transparency since these cells secrete extracellular matrix that is not organized like the native tissue [7]. Furthermore, cornea crystalline production is altered, further reducing transparency. These cell types and their response to injury control the delicate balance between stromal healing and fibrosis and thus scar formation.

A number of topical ointments and eye drops designed to decrease inflammation and reduce scar formation following corneal injuries and keratoplasty are clinically available today [8, 9]. However, these treatments have significant shortcomings [10, 11]. Cellular therapies are emerging as potential therapies to prevent scarring and to reduce the need for expensive and invasive allograft transplantation. In clinical trials, autologous and allogeneic limbal stem cells (LSCs) were transplanted to restore corneal epithelium after ocular surface burns to prevent chronic inflammation and corneal scarring [12, 13]. Mesenchymal stem cells (MSCs), purported to be immunomodulatory and secrete anti-inflammatory molecules, also demonstrated efficacy in reducing corneal scar formation in preclinical models [14-16]. Corneal stromal stem cells transplantation has also shown promising outcomes in reducing corneal fibrosis and scarring in animal studies [17]. While these studies are promising, cell therapies have numerous challenges, including high manufacturing cost and challenges in batch-to-batch reproducibility. Difficulties of application and time sensitivity also arise when considering cell therapies for reducing inflammation and promoting functional wound healing after surgical trauma. Acellular materials such as amniotic membrane and fibrin glue are biological alternatives to cell therapies that demonstrate some efficacy [18, 19]. However, these options have practical challenges that limit efficacy and leads to questionable reproducibility [20, 21].

We have found that Th2 pro-regenerative T cell response can be leveraged for tissue repair. T cells are a key component of the adaptive immune system that is increasingly recognized for their role in wound healing and tissue repair. For example, CD4⁺ lymphocytes, so-called helper T (Th) cells regulate bone, liver and muscle repair processes [22-24]. These cells are notable in their antigen specificity through their variable and diverse T cell receptors (TCRs). Classes of effector cells can also be associated with undesirable outcomes such as allergy and asthma (Th2), autoimmunity, fibrosis, and cancer promotion. Understanding of their role in biomaterial responses is in its infancy. Because the local environment regulates effector functions, non-traditional cytokine profiles of the Th2 responses in the context of trauma, regeneration, and foreign body responses can be expected and engaged in immunotherapy design. Nonclassical lymphocytes, i.e., gamma-delta (γδ) cells and innate lymphoid cells (ILC), can, in parallel, bolster classic T cell responses via production of the same cytokine mediators. Our previous studies revealed the details of pro-regenerative immune responses to biological scaffolds. It has been defined how the biomaterial-dependent, pro-regenerative macrophage phenotype is induced by Th2 T cells [23]. The ECM biomaterial increases the overall quantity of IL4 producing immune cells that enter the wound space specifically increases IL4 producing Th2 T cells, and this specific Th2 response triggered by ECM materials presented a pro-regenerative environment. However, in ocular repair, the role of IL4 is usually associated with allergic reaction or parasite infection, understanding this type of immune response and how it may be modulated to a pro-regenerative phenotype will provide us a novel pathway toward corneal repair and reconstruction.

We now apply immunological techniques to corneal repair, regeneration and healing and treatment of ocular inflammatory diseases and disorders such as dry eye disease.

Without being bound by any theory, according to embodiments of the invention, type 2 immune response drives the pro-regenerative processes in response to synthetic implants. In particular, the urinary derived ECM biomaterial (“UBM-ECM” or “UBM”) increases the overall quantity of IL4 producing immune cells that enter the wound space specifically increases IL4 producing Th2 T cells, and this specific Th2 response triggered by ECM materials presented a pro-regenerative environment. To achieve an ideal ocular repair, the role of IL4 has been investigated in both innate and adaptive immune responses.

### Biocompatible Scaffolds

ECM scaffolds have been prepared from numerous mammalian (allogeneic and xenogeneic) sources (12), however similarly prepared ECM materials elicit comparable functional repair outcomes in many instances (13). Clinical applications include replacement and reconstruction of tissue voids left following tumor resection; mastectomy/lumpectomy following breast cancer, dural repair after meningioma, and re-epithelialization following esophageal cancer resection (14-17). These applications potentially place ECM scaffolds in proximity to residual cancer cells near the margins, and thus a tumor permissive environment may have severe consequences.

In one aspect, a diverse population of immune cells is recruited into scaffolds and the surrounding area, including macrophages, T lymphocytes and B lymphocytes. The scaffolds induced a pro-regenerative type-2 response, characterized by an mTOR/Rictor-dependent T_{H}2 pathway and IL-4-dependent macrophage polarization, which is critical for functional tissue regeneration.

Generally, preferred materials include those that are biocompatible, biodegradable, and have mechanical properties similar to that of native tissue can be used as a scaffold for the treatments, compositions and kits as disclosed herein, including for example elastomeric scaffolds.

In one embodiment, a suitable scaffold comprises a powdered biological extracellular matrix (ECM). In certain embodiments, the ECM is encased in a laminar sheath of ECM. In yet another embodiment, the scaffold comprises particulate ECM derived from porcine urinary bladder (UBM-ECM).

Preferred biocompatible scaffolds herein are pro-regenerative scaffolds and may further comprise one or more immune cell modulating agents. For instance, suitable immune cell modulating agents comprise: cytokines, monokines, chemokines, adjuvants, vaccines, or antigens, or combinations thereof.

In certain embodiments, the biocompatible scaffold comprises one or more other therapeutic agents. In certain embodiments, the scaffold comprises agents to recruit selected cell types, such as stem cells, or induce differentiation of cells. In certain embodiments, combinations of cells and one or more immune cell modulating agents are added to the scaffold before or during implantation in a patient.

More particularly, the biocompatible scaffold may comprise one or more antimicrobial agents. The term antimicrobial agent as used herein refers to an agent that destroys, inhibits and/or prevents the propagation, growth, colonization and multiplication of unwanted organisms. The term “organism” includes, but is not limited to, microorganisms, bacteria, undulating bacteria, spirochetes, spores, spore-forming organisms, gram-negative organisms, gram-positive organisms, yeasts, fungi, molds, viruses, aerobic organisms, anaerobic organisms and mycobacteria. Specific examples of such organisms include the fungi Aspergillusniger, Aspergillusflavus, Rhizopusnigricans, Cladosproriumherbarium, Epidermophytonfloccosum, Trichophytonmentagrophytes, Histoplasmacapsulatum, and the like; bacteria such as Pseudomanasaeruginosa, Escherichiacoli, Proteusvulgaris, Staphylococcusaureus, Staphylococcusepidermis, Streptococcusfaecalis, Klebsiella, Enterobacteraerogenes, Proteusmirabilis, other gram-negative bacteria and other gram-positive bacteria, mycobactin and the like; and yeast such as Saccharomcycescerevisiae, Candidaalbicans, and the like. Additionally, spores of microorganisms, protozoa, mycoplasma, yeast, fungi, viruses and the like are organisms as referred to herein.

More specifically, a biocompatible scaffold may suitably comprise one or more antibacterials such as one or more of Bacitrin, Besifloxacin (e.g. Besivance); (Ciprofloxacin (e.g. Ciloxan); Erythromycin; Gatifloxacin (e.g. Zymar, Tymer); Gentamicin (e.g. Genoptic, Garamycin); Lomefloxacin (e.g. Okacin); Levofloxacin (e.g. Iquix, Quixin); Moxifloxacin (e.g. Vigamox); Ofloxacin (e.g. Oflox, Optiflox); Sulfacetamide (e.g. Bleph-10, Sulf-10); Tobramycin sulfate (e.g. Tobrex); and Tosufloxacin (e.g. Ozex).

A biocompatible scaffold also suitably may comprise one or more antibiotics such as one or more of Amikacin (e.g. Amikacin sulfate); Ampicillin (e.g. Ampicillin sodium); Bacitracin (e.g. Bacitracin zinc); Cefazolin (e.g. Cefazolin sodium); Ceftazidime; Ceftriaxone; Clindamycin; Colistimethate (e.g. Colistimethate sodium); Erythromycin; Gentamicin (e.g. Gentamicin sulfate); Imipenem/cilastatin; Kanamycin (e.g. Kanamycin sulfate); Neomycin (e.g. Neomycin sulfate); Penicillin G; Piperacillin; Polymyxin B sulfate; Ticarcillin (e.g. Ticarcillin disodium); Tobramycin (e.g. Tobramycin sulfate); and Vancomycin (e.g. Vancomycin hydrochloride).

A biocompatible scaffold also suitably may comprise one or more antifungal agents such as one or more of Amphotericin B (e.g. Fungizone ®); Liposomal amphotericin B Fluconazole (e.g. Diflucan ®); Flucytosine (e.g. Ancobon ®); Itraconazole (e.g. Sporanox ®); Ketoconazole (e.g. Nizoral ®); Natamycin (e.g. Natacyn ®); and Voriconazole (e.g. Vfend ®).

A biocompatible scaffold also suitably may comprise one or more antiviral agents such as one or more of Trifluridine (e.g. Viroptic ®); Acyclovir (e.g. Acyclovir sodium); Cidofovir (e.g. Vistide ®); Famciclovir (e.g. Famvir ®); Fomivirsen (e.g. Vitravene ®): Foscarnet such as Foscarnet sodium (e.g. Foscavir ®); Ganciclovir such as Ganciclovir gel (e.g. Zirgan®, Virgan), Ganciclovir sodium (e.g. Cytovene ®, Vitrasert ®); and Valacyclovir (e.g. Valtrex ®).

Historical classification of macrophages defines the M1 phenotype (e.g., CD86⁺ and Nos2, Tnfa expression) and M2 phenotype (e.g., CD206⁺ and Arg1, Fizz1 expression) as opposite poles governing pro-inflammatory and anti-inflammatory or wound-healing responses, respectively. Recent evidence highlights the heterogeneity of macrophage phenotype and the role of multiple macrophage subtypes in cardiac wound healing (Epelman S., et al. Nat Rev Immunol, 2015, 15(2): p. 117-29), scar formation, and outcomes of certain cancers (Lewis, C.E. and J.W. Pollard, Cancer Res, 2006. 66(2): p. 605-12). Macrophage polarization occurs along a spectrum, and a coordinated timing of the differing phenotypes enables clearance of infection followed by healing of damaged tissue. This polarization is mediated by both environmental factors and further, can be modified by signals from cells of the adaptive immune system, particularly T cells. Macrophages and dendritic cells present antigens and activate T cells, which in turn modulate other immune cells through secretion of cytokines. One such cytokine is interleukin 4 (IL-4) (Tidball, J.G. and S.A. Villalta, Am J Physiol Regul Integr Comp Physiol, 2010. 298(5): p. R1173-87; Salmon-Ehr, V., et al., Lab Invest, 2000. 80(8): p. 1337-43).

According to the techniques herein, biomaterials may induce influx of macrophages with a particularly strong M2 phenotype and that this phenotype may be dependent on the adaptive immune system, which is characterized by a T helper 2 (T_{H}2) cell phenotype. The enhanced T_{H}2/M2 response may be associated with a pro-regenerative cytokine environment and anti-tumor responses as described in the examples section which follows.

The scaffolds can comprise any suitable combination of synthetic polymeric components and biological polymeric components. As used herein, the term “polymer” refers to both synthetic polymeric components and biological polymeric components. “Biological polymer(s)” are polymers that can be obtained from biological sources, such as, without limitation, mammalian or vertebrate tissue, as in the case of certain extracellular matrix-derived (ECM-derived) compositions. Biological polymers can be modified by additional processing steps. Polymer(s), in general include, for example and without limitation, mono-polymer(s), copolymer(s), polymeric blend(s), block polymer(s), block copolymer(s), cross-linked polymer(s), non-cross-linked polymer(s), linear-, branched-, comb-, star-, and/or dendrite-shaped polymer(s), where polymer(s) can be formed into any useful form, for example and without limitation, a hydrogel, a porous mesh, a fiber, woven mesh, or non-woven mesh, such as, for example and without limitation, a non-woven mesh formed by electrodeposition.

Generally, the polymeric components suitable for the scaffold described herein may be any polymer that is biodegradable and biocompatible. By “biodegradable”, it is meant that a polymer, once implanted and placed in contact with bodily fluids and/or tissues, will degrade either partially or completely through chemical, biochemical and/or enzymatic processes. Non-limiting examples of such chemical reactions include acid/base reactions, hydrolysis reactions, and enzymatic cleavage. In certain non-limiting embodiments, the biodegradable polymers may comprise homopolymers, copolymers, and/or polymeric blends comprising, without limitation, one or more of the following monomers: glycolide, lactide, caprolactone, dioxanone, and trimethylene carbonate. Non-limiting examples of biodegradeable polymers include poly(ester urethane) urea elastomers (PEUU) and poly(ether ester urethane) urea elastomers (PEEUU). In other non-limiting embodiments, the polymer(s) comprise labile chemical moieties, non-limiting examples of which include esters, anhydrides, polyanhydrides, or amides, which can be useful in, for example and without limitation, controlling the degradation rate of the scaffold and/or the release rate of therapeutic agents from the scaffold. Alternatively, the polymer(s) may contain peptides or biomacromolecules as building blocks which are susceptible to chemical reactions once placed in situ. For example, the polymer is a polypeptide comprising the amino acid sequence alanine-alanine-lysine, which confers enzymatic lability to the polymer. In another non-limiting embodiment, the polymer composition may comprise a biomacromolecular component derived from an ECM. For example, the polymer composition may comprise the biomacromolecule collagen so that collagenase, which is present in situ, can degrade the collagen.

In embodiments, the scaffolds are biocompatible. By “biocompatible,” it is meant that a polymer composition and its normal in vivo degradation products are cytocompatible and are substantially non-toxic and non-carcinogenic in a patient within useful, practical and/or acceptable tolerances. By “cytocompatible,” it is meant that the polymer can sustain a population of cells and/or the polymer composition, device, and degradation products, thereof are not cytotoxic and/or carcinogenic within useful, practical and/or acceptable tolerances. For example, the scaffold when placed in a human epithelial cell culture does not adversely affect the viability, growth, adhesion, and number of cells. In one non-limiting embodiment, the compositions, and/or devices are “biocompatible” to the extent they are acceptable for use in a human patient according to applicable regulatory standards in a given jurisdiction. In another example the biocompatible polymer, when implanted in a patient, does not cause a substantial adverse reaction or substantial harm to cells and tissues in the body, for instance, the polymer composition or device does not cause necrosis or an infection resulting in harm to tissues from the implanted scaffold.

In preferred aspects, the biocompatible scaffold or extracellular matrix comprises and includes an extracellular matrix-derived material.

As used herein, the terms “extracellular matrix” and “ECM” refer to a mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. By “ECM-derived material” it is meant a composition that is prepared from a natural ECM or from an in vitro source wherein the ECM is produced by cultured cells and comprises one or more polymeric components (constituents) of native ECM. ECM preparations can be considered to be “decellularized” or “acellular”, meaning the cells have been removed from the source tissue through processes described herein and known in the art.

In certain embodiments, preferred ECM materials may comprise collagen-based or collagen-containing materials.

According to one non-limiting example of the ECM-derived material, ECM is isolated from a vertebrate animal, for example, from a warm blooded mammalian vertebrate animal including, but not limited to, human, monkey, pig, cow, sheep, etc. The ECM may be derived from any organ or tissue, including without limitation, urinary bladder, intestine, liver, heart, esophagus, spleen, stomach and dermis. The ECM can comprise any portion or tissue obtained from an organ, including, for example and without limitation, submucosa, epithelial basement membrane, tunica propria, etc. In one non-limiting embodiment, the ECM is isolated from urinary bladder, which may or may not include the basement membrane. In another non-limiting embodiment, the ECM includes at least a portion of the basement membrane. In certain non-limiting embodiments, the material that serves as the biological component of the scaffold consists primarily (e.g., greater than 70%, 80%, or 90%) of ECM. In another non-limiting embodiment, the scaffold may contain at least 50% ECM, at least 60% ECM, at least 70% ECM, and at least 80% ECM. In yet another non-limiting embodiment, the biodegradable elastomeric scaffold comprises at least 10% ECM. The ECM material may or may not retain some of the cellular elements that comprised the original tissue such as capillary endothelial cells or fibrocytes. The type of ECM used in the scaffold can vary depending on the intended immune cell or other cell types to be recruited

In one non-limiting embodiment, the ECM is harvested from porcine urinary bladders (also known as urinary bladder matrix or UBM). Briefly, the ECM is prepared by removing the urinary bladder tissue from a pig and trimming residual external connective tissues, including adipose tissue. All residual urine is removed by repeated washes with tap water. The tissue is delaminated by first soaking the tissue in a deepithelializing solution, for example and without limitation, hypertonic saline (e.g. 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. The relatively fragile epithelial basement membrane is invariably damaged and removed by any mechanical abrasion on the luminal surface. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated.

In some embodiments, ECM is prepared as a powder or particles. Such powder can be made according to the method of Gilbert et al., Biomaterials 26 (2005) 1431-1435, herein incorporated by reference in its entirety. For example, UBM sheets can be lyophilized and then chopped into small sheets for immersion in liquid nitrogen. The snap frozen material can then be comminuted so that particles are small enough to be placed in a rotary knife mill, where the ECM is powdered. Similarly, by precipitating NaCl within the ECM tissue the material will fracture into uniformly sized particles, which can be snap frozen, lyophilized, and powdered. The ECM typically is derived from mammalian tissue, such as, without limitation from one of urinary bladder, spleen, liver, heart, pancreas, ovary, or small intestine. In certain embodiments, the ECM is derived from a pig, cow, horse, monkey, or human.

In one aspect, the composition is formulated as a gel. Hydrogels of biological scaffold materials such as an ECM hydrogel may be preferred. The term hydrogel as used herein refers to a substance formed when a polymer (natural or synthetic) becomes a 3-D open-lattice structure that entraps solution molecules, typically water, to form a gel. A polymer may form a hydrogel by, for example, aggregation, coagulation, hydrophobic interactions, cross-linking, salt bridges, etc.

Hydrogels may be prepared for example by extracting an organ material (e.g. pig bladder), decellularizing the extracted organ portion to yield extracellular matrix. The extracellular matrix can be powdered, the resulting powder digested, and the digest reconstituted into a hydrogel. In a particular protocol, lyophilized UBM-ECM is milled to a powder, and digested at 10 mg/ml with 1 mg/ml pepsin for 1-3 days. The resulting digest can be reconstituted into a hydrogel. See also procedures for preparing a hydrogel (including UBM-ECM hydrogels) in Faust et al., J Biomater Appl, 2017. 31(9): p. 1277-1295; Medberry et al., Biomaterials, 2013. 34(4): p. 1033-40).

### Micronization of Tissues

Once the tissues have been dehydrated, the dehydrated tissue(s) is micronized. The micronized compositions can be produced using instruments known in the art. For example, the Retsch Oscillating Mill MM400 can be used to produce the micronized compositions described herein. The particle size of the materials in the micronized composition can vary as well depending upon the application of the micronized composition. In one aspect, the micronized composition has particles that have a mean particle size of less than 100 µm, less than 80 µm, less than 60 µm, less than 50 µm, less than 40 µm, or less than 30 µm. In general, mean particle size will not be less than 5, 4, 3, 2 or 1 µm. For certain aspects, a mean particle size of from 5 µm to 30, 40, 50 or 60 µm will be preferred, or a mean particle size of from 10 µm to 30, 40, 50 or 60 µm.

In one embodiment, micronization is performed by mechanical grinding or shredding. In another aspect, micronization is performed cryogenic grinding. In this aspect, the grinding jar containing the tissue is continually cooled with liquid nitrogen from the integrated cooling system before and during the grinding process. Thus the sample is embrittled and volatile components are preserved. Moreover, the denaturing of proteins in the tissues or tissue layer,

The selection of components used to make the micronized components described herein can vary depending upon the end-use of the composition. For example, bladder, amnion, chorion, etc., or any combination thereof as individual components can be admixed with one another and subsequently micronized. In another aspect, one or more ECMs composed of one or more tissue sources.

In addition to urinary bladder tissue, additional components can be added to the composition prior to and/or after micronization. In one aspect, a filler can be added. Examples of fillers include, but are not limited to, allograft pericardium, allograft acellular dermis, Wharton’s jelly separated from vascular structures (i.e., umbilical vein and artery) and surrounding membrane, purified xenograft Type-1 collagen, biocellulose polymers or copolymers, biocompatible synthetic polymer or copolymer films, purified small intestinal submucosa, bladder acellular matrix, cadaveric fascia, or any combination thereof.

In another embodiment, a bioactive agent can be added to the composition prior to and/or after micronization. Examples of bioactive agents include, but are not limited to, naturally occurring growth factors sourced from platelet concentrates, either using autologous blood collection and separation products, or platelet concentrates sourced from expired banked blood; bone marrow aspirate; stem cells derived from concentrated human placental cord blood stem cells, concentrated amniotic fluid stem cells or stem cells grown in a bioreactor; or antibiotic, immunomodulatory agents and the like. Upon application of the micronized composition with bioactive agent to the region of interest, the bioactive agent is delivered to the region over time. Thus, the micronized particles described herein are useful as delivery devices of bioactive agents and other pharmaceutical agents when administered to a subject. Release profiles can be modified based on, among other things, the selection of the components used to make the micronized compositions as well as the size of the particles.

In certain embodiments, the micronized composition can be used to form a three-dimensional construct. For example, the micronized particles can be treated with a cross-linking agent then placed in a mold having specific dimensions. Alternatively, the micronized particles can be placed into the mold and subsequently treated with the cross-linking agent. In one aspect, the cross-linked particles can be manually formed into any desired shape. In other aspects, one or more adhesives can be admixed with an adhesive prior to being introduced into the mold. Examples of such adhesives include, but are not limited to, fibrin sealants, cyanoacrylates, gelatin and thrombin products, polyethylene glycol polymer, albumin, and glutaraldehyde products. Not wishing to be bound by theory, the three-dimensional construct composed of smaller micronized particles will produce a denser product capable of bearing mechanical loads. Alternatively, larger micronized particles will produce constructs that are less dense and possess compressive properties. This feature can be useful in non-load void filling, especially where it is desirable to have a product that will conform to irregular shapes. The three-dimensional constructs can include one or more bioactive agents described herein.

In certain embodiments, the concentration of the cross-linking agent is from 0.1 M to 5 M, 0.1 M to 4 M, 0.1 M to 3 M, 0.1 M to 2 M, or 0.1 M to 1 M. The cross-linking agent generally possesses two or more functional groups capable of reacting with proteins to produce covalent bonds. In one aspect, the cross-linking agent possesses groups that can react with amino groups present on the protein. Examples of such functional groups include, but are not limited to, hydroxyl groups, substituted or unsubstituted amino groups, carboxyl groups, and aldehyde groups. In one aspect, the cross-linker can be a dialdehyde such as, for example, glutaraldehyde. In another aspect, the cross-linker can be a carbodiimide such as, for example, (N-(3-dimethylaminopropyl)-N′-ethyl-carbodiimide (EDC). In other aspects, the cross-linker can be an oxidized dextran, p-azidobenzoyl hydrazide, N-[alpha-maleimidoacetoxy]succinimide ester, p-azidophenyl glyoxal monohydrate, bis-[beta-(4-azidosalicylamido)ethyl]disulfide, bis-[sulfosuccinimidyl]suberate, dithiobis[succinimidyl]propionate, disuccinimidyl suberate, and 1-ethyl-3-[3-dimethylaminopropyl]carbodiimide hydrochloride, a bifunctional oxirane (OXR), or ethylene glycol diglycidyl ether (EGDE).

In certain embodiments, sugar is the cross-linking agent, where the sugar can react with proteins present in the ECM to form a covalent bond. For example, the sugar can react with proteins by the Maillard reaction, which is initiated by the nonenzymatic glycosylation of amino groups on proteins by reducing sugars and leads to the subsequent formation of covalent bonds. Examples of sugars useful as a cross-linking agent include, but are not limited to, D-ribose, glycerose, altrose, talose, ertheose, glucose, lyxose, mannose, xylose, gulose, arabinose, idose, allose, galactose, maltose, lactose, sucrose, cellibiose, gentibiose, melibiose, turanose, trehalose, isomaltose, or any combination thereof.

In other embodiments, the micronized compositions described herein can be formulated in any excipient the biological system or entity can tolerate to produce pharmaceutical compositions. Examples of such excipients include, but are not limited to, water, aqueous hyaluronic acid, saline, Ringer’s solution, dextrose solution, Hank’s solution, and other aqueous physiologically balanced salt solutions. Nonaqueous vehicles, such as fixed oils, vegetable oils such as olive oil and sesame oil, triglycerides, propylene glycol, polyethylene glycol, and injectable organic esters such as ethyl oleate can also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosol, cresols, formalin and benzyl alcohol. In certain aspects, the pH can be modified depending upon the mode of administration. Additionally, the pharmaceutical compositions can include carriers, thickeners, diluents, preservatives, surface active agents and the like in addition to the compounds described herein.

The pharmaceutical compositions can be prepared using techniques known in the art. In one aspect, the composition is prepared by admixing a micronized composition described herein with a pharmaceutically-acceptable compound and/or carrier. The term “admixing” is defined as mixing the two components together so that there is no chemical reaction or physical interaction. The term “admixing” also includes the chemical reaction or physical interaction between the compound and the pharmaceutically-acceptable compound.

It will be appreciated that the actual preferred amounts of micronized composition in a specified case will vary according to the specific compound being utilized, the particular compositions formulated, the mode of application, and the particular situs and subject being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physician’s Desk Reference, PDR Network (2017).

The pharmaceutical compositions described herein can be administered in a number of ways depending on whether local or systemic treatment is desired, and on the area to be treated. In one aspect, administration can be by injection, where the micronized composition is formulated into a liquid or gel. In other aspects, the micronized composition can be formulated to be applied internally to a subject. In other aspects, the micronized composition can be applied topically, parenterally (e.g., intraocular), local ocularly (e.g. subconjunctivaly, intravitreal, retrobulbar, intracameral), or systemically. In certain embodiments, the micronized compositions can be formulated as a topical composition applied directly to the ocular region or cornea. Formulations for topical administration can include, emulsions, creams, aqueous solutions, oils, ointments, pastes, gels, lotions, milks, foams, suspensions and powders. In one aspect, the topical composition can include one or more surfactants and/or emulsifiers. Surfactants (or surface-active substances) that may be present are anionic, non-ionic, cationic and/or amphoteric surfactants. Typical examples of anionic surfactants include, but are not limited to, soaps, alkylbenzenesulfonates, alkanesulfonates, olefin sulfonates, alkyl ether sulfonates, glycerol ether sulfonates, α-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids, e.g. acyl lactylates, acyl tartrates, acyl glutamates and acyl aspartates, alkyl oligoglucoside sulfates, protein fatty acid condensates (in particular wheat-based vegetable products) and alkyl (ether) phosphates. Examples of non-ionic surfactants include, but are not limited to, fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers or mixed formals, optionally partially oxidized alk(en)yl oligoglycosides or glucoronic acid derivatives, fatty acid N-alkylglucamides, protein hydrolysates (in particular wheat-based vegetable products), polyol fatty acid esters, sugar esters, sorbitan esters, polysorbates and amine oxides. Examples of amphoteric or zwitterionic surfactants include, but are not limited to, alkylbetaines, alkylamidobetaines, aminopropionates, aminoglycinates, imidazolinium-betaines and sulfobetaines.

In certain embodiments, the surfactant can be fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, alpha-olefinsulfonates, ether carboxylic acids, alkyl oligoglucosides, fatty acid glucamides, alkylamidobetaines, amphoacetals and/or protein fatty acid condensates.

In certain embodiments, the emulsifier can be a nonionogenic surfactant selected from the following: addition products of from 2 to 30 mol of ethylene oxide and/or 0 to 5 mol of propylene oxide onto linear fatty alcohols having 8 to 22 carbon atoms, onto fatty acids having 12 to 22 carbon atoms, onto alkylphenols having 8 to 15 carbon atoms in the alkyl group, and onto alkylamines having 8 to 22 carbon atoms in the alkyl radical; alkyl and/or alkenyl oligoglycosides having 8 to 22 carbon atoms in the alk(en)yl radical and the ethoxylated analogs thereof; addition products of from 1 to 15 mol of ethylene oxide onto castor oil and/or hydrogenated castor oil; addition products of from 15 to 60 mol of ethylene oxide onto castor oil and/or hydrogenated castor oil; partial esters of glycerol and/or sorbitan with unsaturated, linear or saturated, branched fatty acids having 12 to 22 carbon atoms and/or hydroxycarboxylic acids having 3 to 18 carbon atoms, and the adducts thereof with 1 to 30 mol of ethylene oxide; partial esters of polyglycerol (average degree of selfcondensation 2 to 8), trimethylolpropane, pentaerythritol, sugar alcohols (e.g. sorbitol), alkyl glucosides (e.g. methyl glucoside, butyl glucoside, lauryl glucoside), and polyglucosides (e.g. cellulose) with saturated and/or unsaturated, linear or branched fatty acids having 12 to 22 carbon atoms and/or hydroxycarboxylic acids having 3 to 18 carbon atoms, and the adducts thereof with 1 to 30 mol of ethylene oxide; mixed esters of pentaerythritol, fatty acids, citric acid and fatty alcohols and/or mixed esters of fatty acids having 6 to 22 carbon atoms, methylglucose and polyols, preferably glycerol or polyglycerol, mono-, di- and trialkyl phosphates, and mono-, di- and/or tri-PEG alkyl phosphates and salts thereof; wool wax alcohols; polysiloxane-polyalkyl-polyether copolymers and corresponding derivatives; and block copolymers, e.g. polyethylene glycol-30 dipolyhydroxystearates. In one aspect, the emulsifier is a polyalkylene glycol such as, for example, polyethylene glycol or polypropylene glycol. In another aspect, the emulsifier is polyethylene glycol having a molecular weight 100 Da to 5,000 Da, 200 Da to 2,500 Da, 300 Da to 1,000 Da, 400 Da to 750 Da, 550 Da to 650 Da, or about 600 Da.

In certain embodiments, the emulsifier is composed of one or more fatty alcohols. In one aspect, the fatty alcohol is a liner or branched C₆ to C₃₅ fatty alcohol. Examples of fatty alcohols include, but are not limited to, capryl alcohol (1-octanol), 2-ethyl hexanol, pelargonic alcohol (1-nonanol), capric alcohol (1-decanol, decyl alcohol), undecyl alcohol (1-undecanol, undecanol, hendecanol), lauryl alcohol (dodecanol, 1-dodecanol), tridecyl alcohol (1-tridecanol, tridecanol, isotridecanol), myristyl alcohol (1-tetradecanol), pentadecyl alcohol (1-pentadecanol, pentadecanol), cetyl alcohol (1-hexadecanol), palmitoleyl alcohol (cis-9-hexadecen-1-ol), heptadecyl alcohol (1-n-heptadecanol, heptadecanol), stearyl alcohol (1-octadecanol), isostearyl alcohol (16-methylheptadecan-1-ol), elaidyl alcohol (9E-octadecen-1-ol), oleyl alcohol (cis-9-octadecen-1-ol), linoleyl alcohol (9Z, 12Z-octadecadien-1-ol), elaidolinoleyl alcohol (9E, 12E-octadecadien-1-ol), linolenyl alcohol (9Z, 12Z, 15Z-octadecatrien-1-ol) elaidolinolenyl alcohol (9E, 12E, 15-E-octadecatrien-1-ol), ricinoleyl alcohol (12-hydroxy-9-octadecen-1-ol), nonadecyl alcohol (1-nonadecanol), arachidyl alcohol (1-eicosanol), heneicosyl alcohol (1-heneicosanol), behenyl alcohol (1-docosanol), erucyl alcohol (cis-13-docosen-1-ol), lignoceryl alcohol (1-tetracosanol), ceryl alcohol (1-hexacosanol), montanyl alcohol, cluytyl alcohol (1-octacosanol), myricyl alcohol, melissyl alcohol (1-triacontanol), geddyl alcohol (1-tetratriacontanol), or cetearyl alcohol.

In certain embodiments, the carrier used to produce the topical composition is a mixture polyethylene and one or more fatty alcohols. For example, the carrier is composed of 50% to 99% by weight, 75% to 99% by weight, 90% to 99% by weight, or about 95% by weight polyethylene glycol and 1% to 50% by weight, 1% to 25% by weight, 1% to 10% by weight, or about 5% by weight fatty alcohol. In a further aspect, the carrier is a mixture of polyethylene glycol and cetyl alcohol.

The topical compositions can also include additional components typically present in such compositions. In one aspect, the topical composition can include one or more of the following components: fats, waxes, pearlescent waxes, bodying agents, thickeners, superfatting agents, stabilizers, polymers, silicone compounds, lecithins, phospholipids, biogenic active ingredients, deodorants, antimicrobial agents, antiperspirants, swelling agents, insect repellents, hydrotropes, solubilizers, preservatives, perfume oils and dyes. Examples of each of these components are disclosed in U.S. Pat. No. 8,067,044, which is incorporated by reference with respect these components.

The topical compositions composed of the micronized compositions described herein can be prepared by mixing the particles with the carrier for a sufficient time such that the particles are evenly dispersed throughout the carrier. In the case when the carrier is composed of two or more components, the components can be admixed with one another prior to the addition of the micronized composition. The amount of micronized composition present in the topical composition can vary depending upon the application. In one aspect, the micronized composition is from 0.5% to 20%, 1% to 10%, 2% to 5%, or about 3% by weight of the topical composition.

### Pharmaceutical Therapeutics

In other embodiments, agents discovered to have immunomodulatory activity that enhances anti-tumor immune responses using the methods described herein are useful as a drug or as information for structural modification of existing compounds, e.g., by rational drug design. Such methods are useful for screening agents having an effect on a neoplasia.

For therapeutic uses, the compositions or agents identified using the methods disclosed herein may be administered systemically or locally to a subject for example to facilitate repair or regeneration of corneal tissue. Such agents may also be incorporated directly into a biomaterial scaffold as disclose herein.

Preferable systemic routes of administration include, for example, topical, parenteral (e.g., intraocular), local ocular (e.g. subconjunctival, intravitreal, retrobulbar, intracameral), or systemic application or injections that provide continuous, sustained levels of the drug in the patient.

Treatment of human patients or other animals will be carried out using a therapeutically effective amount of a therapeutic composition as disclosed herein suitably in a physiologically-acceptable carrier. Suitable carriers and their formulation are described, for example, in Remington’s Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with wound healing/tissue regeneration, although in certain instances lower amounts will be needed because of the increased specificity of the compound.

### Formulation of Pharmaceutical Compositions

The administration of an agent or compound or a combination of agents/compounds for the treatment of a wound may be by any suitable means that results in a concentration of the therapeutic that, combined with other components, is effective in ameliorating, reducing, or stabilizing a neoplasia. The compound may be contained in any appropriate amount in any suitable carrier substance, and is generally present in an amount of 1-95% by weight of the total weight of the composition. The composition may be provided in a dosage form that is suitable for topical, parenteral (e.g., intraocular), local ocular (e.g. subconjunctival, intravitreal, retrobulbar, intracameral), or systemic administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

As discussed, eye drops are a preferred route of administration. For an eye drop formulation, one or more therapeutic agents (e.g. biocompatible scaffold that comprises extracellular matrix material(s) such as urinary bladder matrix (UBM)) may be admixed with one or more suitable additives such as a buffer reagent (such as, phosphate buffered saline buffer, borate buffer solution, citrate buffer, tartrate buffer, acetate buffer, amino acid, sodium-acetate, Trisodium Citrate etc.), (such as, carbohydrate, as sorbyl alcohol for isotonicity, glucose and N.F,USP MANNITOL, polyvalent alcohol, as glycerol, concentrated glycerol, polyoxyethylene glycol and propylene glycol, salt, as sodium-chlor), antiseptic-germicide or sanitas (such as, benzalkonium chloride, benzethonium chloride, p-Oxybenzene manthanoate, Oxybenzene manthanoate as p-in methyl or the p-Oxybenzene manthanoate of ethyl, phenylcarbinol, phenylethyl alcohol, Sorbic Acid or its salt, Thiomersalate, butylene-chlorohydrin etc.), solubilizing acid or stablizer (such as, cyclodextrin and derivative thereof, water-soluble polymers, as polyvinylpyrrolidone), tensio-active agent, as Polysorbate 80 (tween 80)), pH value regulator (such as, hydrochloric acid, acetic acid, phosphoric acid, sodium hydroxide, potassium hydroxide, ammonium hydroxide etc.), sequestrant (e.g., sodium ethylene diamine tetracetate, Trisodium Citrate, concentrated phosphoric acid sodium) etc.

Eye ointment compositions suitably may comprise one or more therapeutic agents as disclosed herein together with an ointment base material such as one or more of pure sheep oil, Vaseline, white oil and poly compound ointment base, Liquid Paraffin, polyoxyethylene glycol etc.

Human dosage amounts can initially be determined by extrapolating from the amount of compound used in mice, as a skilled artisan recognizes it is routine in the art to modify the dosage for humans compared to animal models. In certain embodiments it is envisioned that the dosage may vary from between about 1 µg compound/kg body weight to about 5000 mg compound/ kg body weight; or from about 5 mg/ kg body weight to about 4000 mg/ kg body weight or from about 10 mg/ kg body weight to about 3000 mg/ kg body weight; or from about 50 mg/ kg body weight to about 2000 mg/ kg body weight; or from about 100 mg/ kg body weight to about 1000 mg/ kg body weight; or from about 150 mg/ kg body weight to about 500 mg/ kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/ kg body weight. In other embodiments, it is envisaged that doses may be in the range of about 5 mg compound/ kg body to about 20 mg compound/ kg body. In other embodiments the doses may be about 8, 10, 12, 14, 16 or 18 mg/ kg body weight. Of course, this dosage amount may be adjusted upward or downward, as is routinely done in such treatment protocols, depending on the results of the initial clinical trials and the needs of a particular patient.

Pharmaceutical compositions according to the disclosure may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Methods of Treatment

In one preferred aspect, the present disclosure provides a method of repairing and/or reconstructing cornea in a subject. The methods involve administering to a subject in need thereof, an effective amount of agents (or therapeutic agents) of the disclosure. For example, the agents including an effective amount of tissue matrix particles or ECM, or a biocompatible scaffold including the same, can increase IL4 and/or producing T_{H}2 cells. Alternatively, the agents including an effective amount of tissue matrix particles or ECM, or a biocompatible scaffold including the same, that can promote type 2 response modulated by T_{H}2 T cells, for example, via the IL4 production. Preferably, such agents are administered as part of a composition additionally comprising a pharmaceutically acceptable carrier. In a further preferable method, such agents may be applied to, or incorporated into, a biomaterial scaffold.

In one preferred aspect, the present disclosure provides a method of reducing corneal scarring in a subject. The methods involve administering to a subject in need thereof, an effective amount of agents (or therapeutic agents) of the disclosure. For example, the agents including an effective amount of tissue matrix particles or ECM, or a biocompatible scaffold including the same, can increase IL4 and/or producing T_{H}2 cells. Alternatively, the agents including an effective amount of tissue matrix particles or ECM, or a biocompatible scaffold including the same, that can promote type 2 response modulated by T_{H}2 T cells, for example, via the IL4 production. Preferably, such agents are administered as part of a composition additionally comprising a pharmaceutically acceptable carrier. In a further preferable method, such agents may be applied to, or incorporated into, a biomaterial scaffold.

In one preferred aspect, the present disclosure provides a method of promoting or inducing healing of a corneal wound or injury in a subject. The methods involve administering to a subject in need thereof, an effective amount of agents (or therapeutic agents) of the disclosure. For example, the agents including an effective amount of tissue matrix particles or ECM, or a biocompatible scaffold including the same, can increase IL4 and/or producing T_{H}2 cells. Alternatively, the agents including an effective amount of tissue matrix particles or ECM, or a biocompatible scaffold including the same, that can promote type 2 response modulated by T_{H}2 T cells, for example, via the IL4 production. Preferably, such agents are administered as part of a composition additionally comprising a pharmaceutically acceptable carrier. In a further preferable method, such agents may be applied to, or incorporated into, a biomaterial scaffold.

In one preferred aspect, the present disclosure provides a method of preventing fibrosis in a cornea in a subject. The methods involve administering to a subject in need thereof, an effective amount of agents (or therapeutic agents) of the disclosure. For example, the agents including an effective amount of tissue matrix particles or ECM, or a biocompatible scaffold including the same, can increase IL4 and/or producing T_{H}2 cells. Alternatively, the agents including an effective amount of tissue matrix particles or ECM, or a biocompatible scaffold including the same, that can promote type 2 response modulated by T_{H}2 T cells, for example, via the IL4 production. Preferably, such agents are administered as part of a composition additionally comprising a pharmaceutically acceptable carrier. In a further preferable method, such agents may be applied to, or incorporated into, a biomaterial scaffold.

In a further preferred aspect, the present disclosure provides a method of changing immune profile after cornea trauma in a subject. The methods involve administering to a subject in need thereof, an effective amount of agents (or therapeutic agents) of the disclosure. For example, the agents including an effective amount of tissue matrix particles or ECM, or a biocompatible scaffold including the same, can increase IL4 and/or producing T_{H}2 cells. Alternatively, the agents including an effective amount of tissue matrix particles or ECM, or a biocompatible scaffold including the same, that can promote type 2 response modulated by T_{H}2 T cells, for example, via the IL4 production. Preferably, such agents are administered as part of a composition additionally comprising a pharmaceutically acceptable carrier. In a further preferred method, such agents may be applied to, or incorporated into, a biomaterial scaffold.

In yet a preferred aspect, the present disclosure provides a method of treating an ocular inflammatory disease or disorder in a subject. The methods involve administering to a subject in need thereof, an effective amount of agents (or therapeutic agents) of the disclosure. For example, the agents including an effective amount of tissue matrix particles or ECM, or a biocompatible scaffold including the same, can increase IL4 and/or producing T_{H}2 cells. Alternatively, the agents including an effective amount of tissue matrix particles or ECM, or a biocompatible scaffold including the same, that can promote type 2 response modulated by T_{H}2 T cells, for example, via the IL4 production. Preferably, such agents are administered as part of a composition additionally comprising a pharmaceutically acceptable carrier. In a further preferred method, such agents may be applied to, or incorporated into, a biomaterial scaffold. As discussed specific diseases and disorders for treatment include uveitis, severe conjunctivitis (vernal keratoconjunctivitis), and dry eye syndrome (including, but not limited to, Keratoconjunctivitis sicca and Sjogren’s syndrome), diabetic retinopathy, and/or autoimmune ocular inflammatory disease.

In certain embodiments, a biocompatible scaffold and one or more immune cell modulating agents are administered to the subject. The immune cell modulating agents comprise: cytokines, monokines, chemokines, adjuvants, vaccines, antigens, chemotherapeutic agents or combinations thereof.

In certain embodiments, treatment includes administering to the subject urinary bladder matrix (UBM) with different particle sizes, for example, micro- and nano-UBM particles. In some embodiments, the UBM particles may have a size ranging from about 0.1 µm to about 30, 40, 50 or 60 µm, preferably from about 1 µm to about 30, 40, 50 or 60 µm. In some embodiments, the UBM particles may have a size ranging from about 1 µm to about 30, 40, 50 or 60 µm, preferably from about 10 µm to about 30, 40, 50 or 60 µm.

Other embodiments include any of the methods herein wherein the subject is identified as in need of the indicated treatment.

### Combination Therapies

Compositions of the invention may be combined in a pharmaceutical combination formulation, or dosing regimen as combination therapy, with a second compound, for example, chemotherapeutic agents, agents used in the treatment of autoimmune diseases, etc. The second compound of the pharmaceutical combination formulation or dosing regimen preferably has complementary activities to the compounds of the invention such that they do not adversely affect the other(s). Such molecules are suitably present in combination in amounts that are effective for the purpose intended.

The combination therapy may be administered as a simultaneous or sequential regimen. When administered sequentially, the combination may be administered in two or more administrations. The combined administration includes coadministration, using separate formulations or a single pharmaceutical formulation, and consecutive administration in either order, wherein preferably there is a time period while both (or all) active agents simultaneously exert their biological activities. Suitable dosages for any of the above coadministered agents are those presently used and may be lowered due to the combined action (synergy) of the newly identified agent and other chemotherapeutic agents or treatments.

The combination therapy may provide “synergy” and prove “synergistic”, e.g. the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g. by different injections in separate syringes. In general, during alternation therapy, an effective dosage of each active ingredient is administered sequentially, e.g. serially, whereas in combination therapy, effective dosages of two or more active ingredients are administered together.

As an example, the agent may be administered in combination with [to discuss].

The subjects can also be administered the agent in combination with non-surgical

According to the methods of the invention, the agents of the invention may be administered prior to, concurrent with, or following the other therapeutic compounds or therapies. The administration schedule may involve administering the different agents in an alternating fashion. In other embodiments, the agent may be delivered before and during, or during and after, or before and after treatment with other therapies. In some cases, the agent is administered more than 24 hours before the administration of the second agent treatment. In other embodiments, more than one anti-proliferative therapy or an autoimmune therapy may be administered to a subject. For example, the subject may receive the agents of the invention, in combination with both surgery and at least one other anti-proliferative compound. Alternatively, the agent may be administered in combination with more than one therapeutic agent.

### Kits or Pharmaceutical Systems

The present compositions may be assembled into kits or pharmaceutical systems for use in induce a desired response. Kits or pharmaceutical systems according to this aspect of the disclosure comprise a carrier means, such as a box, carton, tube or the like, having in close confinement therein one or more container means, such as vials, tubes, ampoules, bottles and the like. The kits or pharmaceutical systems of the disclosure may also comprise associated instructions for using the agents of the disclosure. Kits of the disclosure include at least one or more biomaterial scaffolds. The kit may include instructions for administering the immunomodulatory agent in combination with one or more additional, distinct therapeutic agents.

The invention has been described in detail with reference to preferred embodiments thereof. However, it will be appreciated that those skilled in the art, upon consideration of this disclosure, may make modifications and improvements within the spirit and scope of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

The examples including the following data include regenerative immunotherapies by using biological scaffold as a type 2 immune agonist to promote cornea repair.

### Example 1: The Immune Response to Cornea Damage

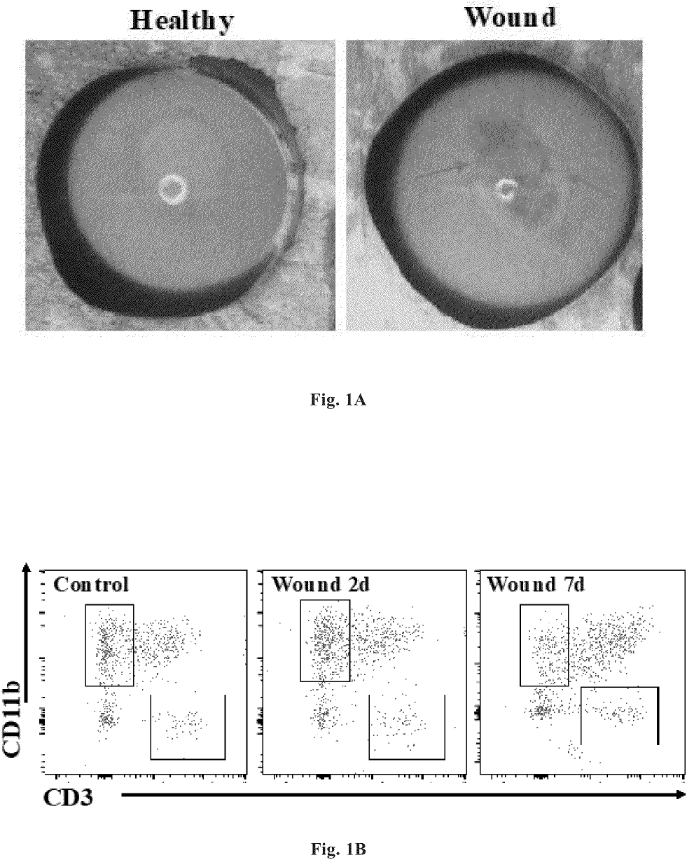

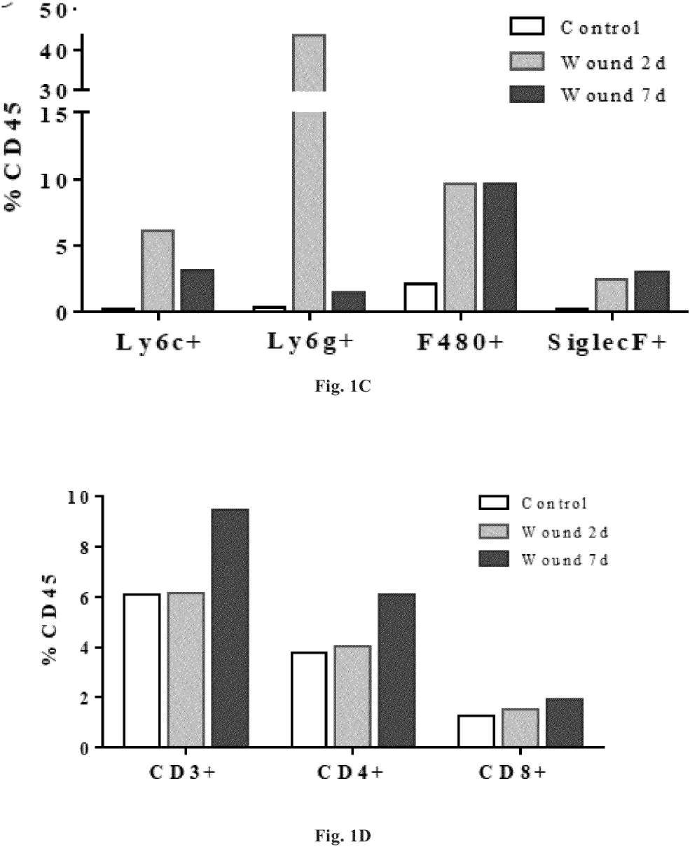

When the cornea is damaged, a complex cascade is initiated involving multiple cell types that produce cytokines that regulate the balance of tissue repair versus fibrosis. Our preliminary data demonstrates the complexity of the innate and adaptive immune response to corneal damage. The immune profile has been evaluated after creating a debridement wound (1.5 mm) on a murine cornea using a flat blade that formed a haze after 14 days (FIG. 1A). Multi-parametric flow cytometry analysis revealed CD11b+ myeloid cells and CD3+ T cells infiltrated into the wounded cornea (FIG. 1B). After 2 days, over 40% of the infiltrating CD11b+ cells were Ly6g+ neutrophils which then decreased by day 7 (FIG. 1C). Macrophages (F480+) and eosinophils (SiglecF+) increased and remained high. Also identified were CD3+ T cells in the wounded corneas 7 days after injury (FIG. 1D).

### Example 2: Modulating the Immune Environment Toward Type 2 Response Reduces Scar Formation in a Murine Corneal Wound.

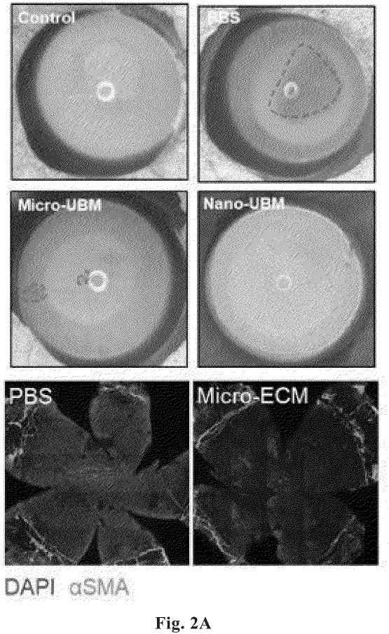

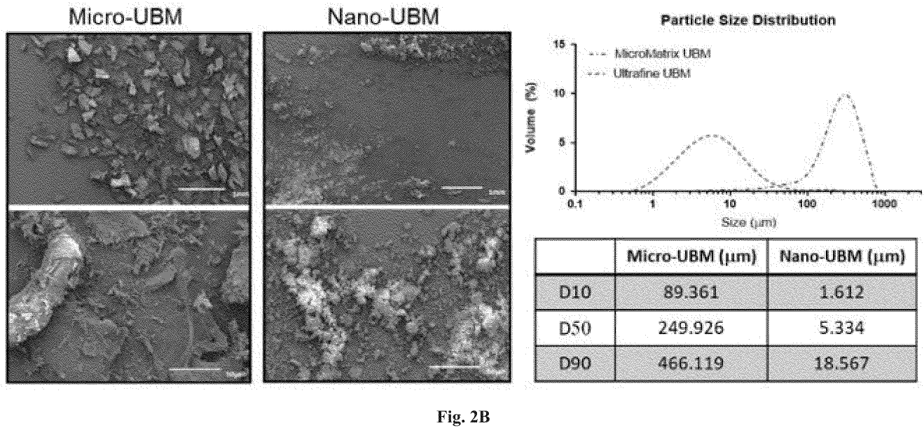

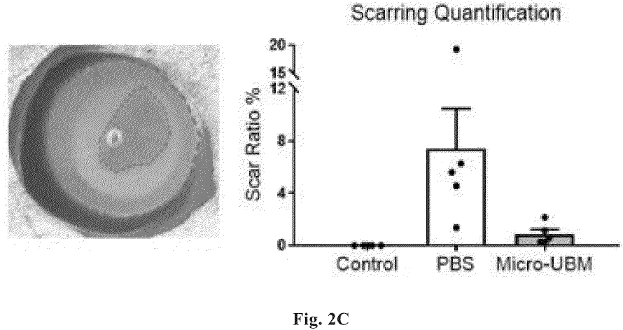

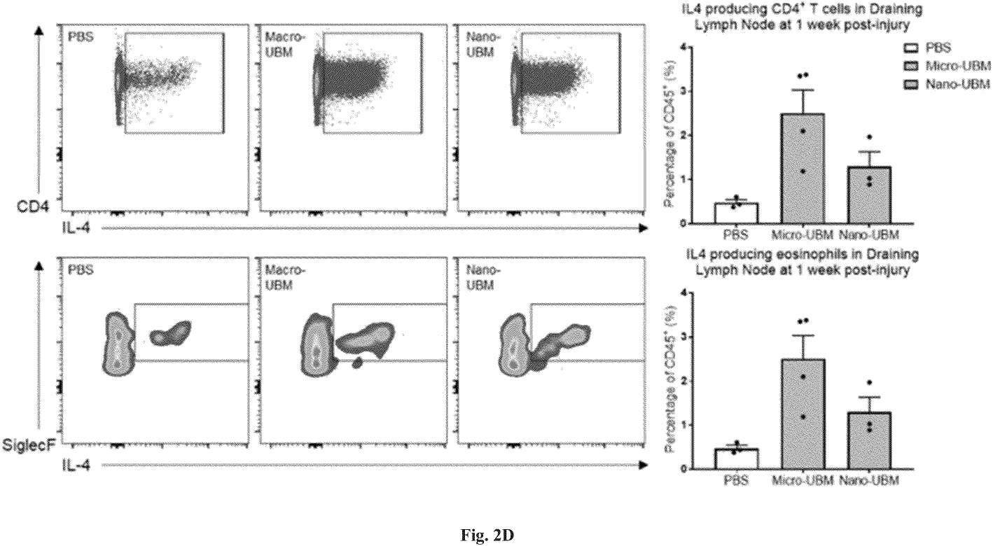

IL4 has been demonstrated to have relevance to tissue repair in multiple tissues including muscle, articular cartilage, liver, heart, lung, and tissues in the central nervous system [29-33]. It is also tested with tissue-derived extracellular matrix from urinary bladder matrix (UBM) with different particle sizes. UBM particles promote type 2 response modulated by T_{H}2 T cells, which results in a pro-regenerative phenotype via the IL4 production [36]. Microparticle UBM and nanoparticle UBM were delivered to the wounded eye by subconjunctival injection. Treatment reduced scarring as demonstrated by immunostaining of the myofibroblast marker α-SMA (alpha smooth muscle actin) compared to saline treated controls (FIG. 2A). Scanning Electron Microscope images showed difference in size and morphology of microparticle UBM and nanoparticle UBM. Particle size analysis compared the diameter differences between micro- and nano-UBM particles (FIG. 2B). Image analysis quantified the scar ratio and demonstrated a significant decrease with different particle size of UBM (FIG. 2C). Flow cytometry of the draining (submandibular) lymph nodes of the wounded cornea (FIG. 2D) revealed a significant increase in IL4 production by T (T_{H}2) cells and eosinophils using an IL4-GFP (green fluorescent protein) reporter mouse strain (4Get) [37].

### Example 3: T_{H}2 Agonist Reduces Fibroblast Recruitment.

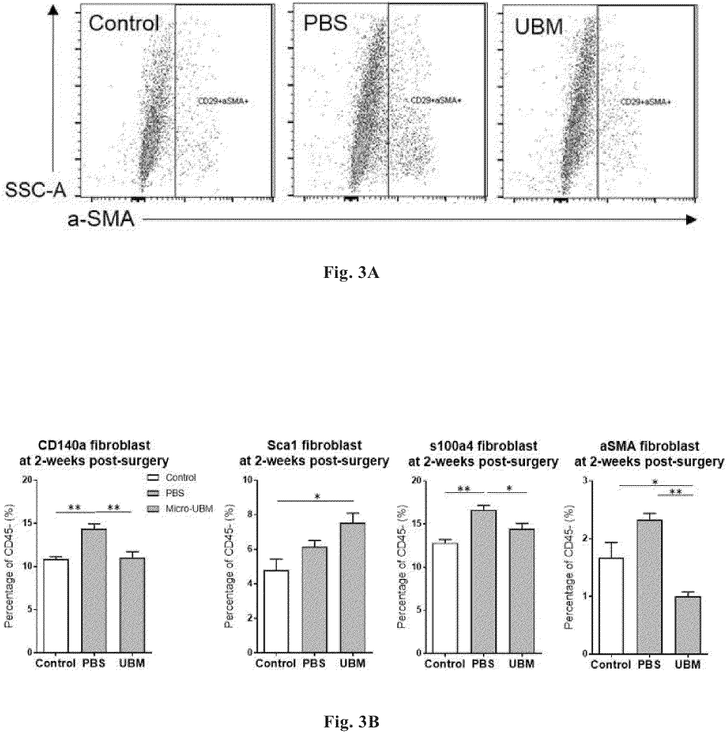

It is important to consider the role of fibroblast diversity, which can play a significant role in scar formation. Inflammatory cytokines from the wound sites could alter fibroblasts’ activities, which eventually leading to pathologically increased fibrosis in the cornea. Preliminary data supports the notion that Th2 agonist can also alter fibroblast heterogeneity. While Seal fibroblast increased with the treatment, CD140a⁺, S100a4⁺, and αSMA⁺ fibroblasts all reduced compared to PBS control (FIGS. 3).

### Methods

### Murine Corneal Wound Model

Mouse corneal debridement wounds can be made as described by the Stepp group, adapted from previous studies [37]. Male mice (~8-week-old BALB/C) can be placed under general anesthesia with ketamine and xylazine via intraperitoneal injection, and atropine ophthalmic ointment can be applied. After 5 min, proparacaine ophthalmic ointment can be applied to the cornea. A 1.5 mm trephine can be used to mark the center of the cornea, following which a flat 1.5 mm blade can be used to remove the epithelial layers and basement membrane. PBS can then be applied to keep the cornea moist. Mice can be injected with painkillers. For subconjunctival injections, 50 µl of PBS vehicle or Th2 agonist can be injected to the subconjunctival space. For eyedrop delivery, PBS vehicle or Th2 agonist can be applied to the ocular surface one time per day for 7 days.

### Flow Cytometry of Immune Cell and Fibroblast Panel

Single cell solutions isolated from the corneas can be stained with immune marker panels. Immune cell recruitment can be monitored with the FACS panels in Table 3, and corneal fibroblast panel is shown in Table 4. Selected Immune cell or fibroblast populations can be sorted for single cell analysis. The systemic immune response can be monitored analyzing draining lymph nodes. Lymph nodes can be harvested and stained using the lymphoid markers to assess T cell proliferation.

TABLE 3

Flow cytometric immune cell characterization

| Lymphoid | | Myeloid | |
|---|---|---|---|
| Marker | Description of target | Marker | Description of target |
| CD45 | Immune Cells | CD45 | Immune cells |
| CD3 | All T cells | NK1.1 | Natural killer cells |
| CD4 | Helper T cells | F4/80 | Macrophages |
| CD8 | Cytotoxic T lymphocytes | CD11b | Myeloid cells |
| FoxP3 | Regulatory T Cells | Ly6g | Neutrophils |
| IL-4 | Th2 T cells | Ly6c | Monocytes |
| IFNγ | Th1 T cells | SiglecF | Eosinophils |

TABLE 4

Flow cytometric fibroblast characterization
Fibroblast

| | |
|---|---|
| Maker | Description of target |
| CD34 | Stem cells |
| CD31 | Endothelial cells |
| CD90 | Keratinocytes |
| CD29 | Fibroblasts |
| αSMA | Myofibroblasts |
| Sca-1 | Fibroblastic progenitor cells |

### Single-Cell Encapsulation and Library Generation

After sorting of immune cells (CD45+) and corneal fibroblasts (CD34-CD31-CD45-CD29⁺), single cells were encapsulated in water-in-oil emulsion along with gel beads coated with unique molecular barcodes using the 10x Genomics Chromium Single-Cell Platform. For single-cell RNA library generation, the manufacturers’ protocol was performed (10× Single Cell 3’ v2). Sequencing was performed using an Illumina HiSeq2500 Rapid Mode with 310 million reads per sample and a sequencing configuration of 26 × 8 × 98 (UMI × Index × Transcript read). The Cell Ranger pipeline software was used to align reads and generate expression matrices for downstream analysis

### Gene Expression Analysis

To extract RNA, the treated and control corneas are dissected and immediately transferred to liquid nitrogen and then pulverized with tissue homogenizer. Using the Qiagen RNA isolation RNeasy systems, mRNA can be extracted using TriZOL reagent and reverse transcribed to cDNA using Super-Script IV VILO Master Mix transcriptase system following the manufacturer’s protocol (Invitrogen, Carlsbad, CA). Real-time PCRs can be performed using StepOnePlus Real Time PCR System with the SYBR Green PCR Master Mix and TaqMan Master Mix. fibrosis genes to be tested include Type I collagen (Col I), Type III collagen (Col III), VEGF and αSMA. Immune related genes include TGF-β, IL1β and IL4. All genes are normalized to β-actin, β2m, and Gapdh.

### Histological Analysis

Wounded corneas and adjacent conjunctival tissue can be imaged and histologically processed by paraffin embedding by using standard techniques using 5 µm-sections. Histological staining can include hematoxylin and eosin (H&E), and Masson’s trichrome to asses collagen deposition over time. The corneas can be the primary focus, along with secondary lymphatic tissues to assess immune responses. Additional immunostaining of immune markers can be performed as described in the preliminary data. Furthermore, if new cellular markers or expression patterns are discovered in the gene expression analysis, additional immunostaining can be performed to confirm protein expression and anatomical location.

In addition to standard histopathologic staining (i.e. H&E), multiplex fluorescent immunohistochemistry (IHC) can be performed, allowing staining for up to 6 markers per histologic cross section using the tyramide amplification system. Fully stained sections can be imaged using the Vectra multispectral imaging system (Vectra 3.0 Automated Quantitative Pathology Imaging System, Perkikn-Elmer). Specific fluorescent signal for each marker can be deconvolved using a spectral library generated for each dye and tissue type, thus removing spectral overlap between dyes and tissue autofluorescence. Cell density, co-localization, spatial distribution of different cell types can be quantified using the automated IiForm image analysis software (Perkin-Elmer).

### Animal Number Calculations

A priori statistical calculations were used to determine the appropriate sample size for each group in the proposed experiments. Based on the independent two-sample Student’s t-test, a total of 4 mice per experimental group can be sufficient for our outcome measures of gene expression, histological analysis, and gross observations (two-tail test, desired statistical power level of 0.9, and significance level of 0.05). All data can be analyzed using the Student’s t-test to determine if significant differences are observed between experimental and control groups at the chosen levels of induction and at each time point. Pain studies can include more mice as multiple time points can be combined for each analysis.

### Reference

Y. Komai, T. Ushiki, The three-dimensional organization of collagen fibrils in the human cornea and sclera, Investigative ophthalmology & visual science 32(8) (1991) 2244-2258.
T. Koller, M. Mrochen, T. Seiler, Complication and failure rates after corneal crosslinking, Journal of Cataract & Refractive Surgery 35(8) (2009) 1358-1362.
A.V. Ljubimov, M. Saghizadeh, Progress in corneal wound healing, Progress in retinal and eye research 49 (2015) 17-45.
W.J. Dupps Jr, S.E. Wilson, Biomechanics and wound healing in the cornea, Experimental eye research 83(4) (2006) 709-720.
S. Li, B. Li, H. Jiang, Y. Wang, M. Qu, H. Duan, Q. Zhou, W. Shi, Macrophage depletion impairs corneal wound healing after autologous transplantation in mice, PloS one 8(4) (2013) e61799.
J.V. Jester, J. Huang, P.A. Barry-Lane, W.W. Kao, W.M. Petroll, H.D. Cavanagh, Transforming growth factorβ-mediated corneal myofibroblast differentiation requires actin and fibronectin assembly, Investigative ophthalmology & visual science 40(9) (1999) 1959-1967.
M.E. Fini, Keratocyte and fibroblast phenotypes in the repairing cornea, Progress in retinal and eye research 18(4) (1999) 529-551.
S.S. Tuli, G.S. Schultz, D.M. Downer, Science and strategy for preventing and managing corneal ulceration, The ocular surface 5(1) (2007) 23-39.
N.G. Soni, A. Pillar, J. Margo, B.H. Jeng, Management of the Persistent Corneal Epithelial Defect, Ocular Surface Disease, Springer2018, pp. 221-231.
P.S. Severn, S.G. Fraser, Bilateral cataracts and glaucoma induced by long-term use of oral prednisolone bought over the internet, The Lancet 368(9535) (2006) 618.
T.O. McDonald, A.R. Borgmann, M.D. Roberts, L.G. Fox, Corneal wound healing: I. Inhibition of stromal healing by three dexamethasone derivatives, Investigative Ophthalmology & Visual Science 9(9) (1970) 703-709.
S. Shukla, S.S. Shanbhag, F. Tavakkoli, S. Varma, V. Singh, S. Basu, Limbal Epithelial and Mesenchymal Stem Cell Therapy for Corneal Regeneration, Current eye research (2019) 1-13.
T.A. Blenkinsop, B. Corneo, S. Temple, J.H. Stern, Ophthalmologic stem cell transplantation therapies, Regenerative medicine 7(6s) (2012) 32-39.
L. Yao, H. Bai, Mesenchymal stem cells and corneal reconstruction, Molecular vision 19 (2013) 2237.
L. Zhang, V.J. Coulson-Thomas, T.G. Ferreira, W.W. Kao, Mesenchymal stem cells for treating ocular surface diseases, BMC ophthalmology, BioMed Central, 2015, p. 155.
D. Almaliotis, G. Koliakos, E. Papakonstantinou, A. Komnenou, A. Thomas, S. Petrakis, I. Nakos, E. Gounari, V. Karampatakis, Mesenchymal stem cells improve healing of the cornea after alkali injury, Graefe’s Archive for Clinical and Experimental Ophthalmology 253(7) (2015) 1121-1135.
S. Basu, A.J. Hertsenberg, M.L. Funderburgh, M.K. Burrow, M.M. Mann, Y. Du, K.L. Lathrop, F.N. Syed-Picard, S.M. Adams, D.E. Birk, Human limbal biopsy-derived stromal stem cells prevent corneal scarring, Science translational medicine 6(266) (2014) 266ra172-266ra172.
V.S. Sangwan, S. Basu, G.K. Vemuganti, K. Sejpal, S.V. Subramaniam, S. Bandyopadhyay, S. Krishnaiah, S. Gaddipati, S. Tiwari, D. Balasubramanian, Clinical outcomes of xeno-free autologous cultivated limbal epithelial transplantation: a 10-year study, British Journal of Ophthalmology 95(11) (2011) 1525-1529.
A. Yeung, L. Faraj, O. McIntosh, V. Dhillon, H. Dua, Fibrin glue inhibits migration of ocular surface epithelial cells, Eye 30(10) (2016) 1389.
I. Rahman, D. Said, V. Maharajan, H. Dua, Amniotic membrane in ophthalmology: indications and limitations, Eye 23(10) (2009) 1954.
H.S. Dua, V.S. Maharajan, A. Hopkinson, Controversies and limitations of amniotic membrane in ophthalmic surgery, Cornea and External Eye Disease, Springer2006, pp. 21-33.
G.S. Baht, L. Vi, B.A. Alman, The role of the immune cells in fracture healing, Current osteoporosis reports 16(2) (2018) 138-145.
K. Sadtler, K. Estrellas, B.W. Allen, M.T. Wolf, H. Fan, A.J. Tam, C.H. Patel, B.S. Luber, H. Wang, K.R. Wagner, Developing a pro-regenerative biomaterial scaffold microenvironment requires T helper 2 cells, Science 352(6283) (2016) 366-370.
C.M. Minutti, L.H. Jackson-Jones, B. García-Fojeda, J.A. Knipper, T.E. Sutherland, N. Logan, E. Ringqvist, R. Guillamat-Prats, D.A. Ferenbach, A. Artigas, Local amplifiers of IL-4Rα-mediated macrophage activation promote repair in lung and liver, Science 356(6342) (2017) 1076-1080.
Z. Tan, X. Qian, R. Jiang, Q. Liu, Y. Wang, C. Chen, X. Wang, B. Ryffel, B. Sun, IL-17A plays a critical role in the pathogenesis of liver fibrosis through hepatic stellate cell activation, The Journal of Immunology 191(4) (2013) 1835-1844.
M. Brodlie, M. McKean, G. Johnson, A. Anderson, C. Hilkens, A. Fisher, P. Corris, J. Lordan, C. Ward, Raised interleukin-17 is immunolocalised to neutrophils in cystic fibrosis lung disease, European Respiratory Journal 37(6) (2011) 1378-1385.
S.K. Chauhan, R. Dana, Role of Th17 cells in the immunopathogenesis of dry eye disease, Mucosal immunology 2(4) (2009) 375.
H. Chen, W. Wang, H. Xie, X. Xu, J. Wu, Z. Jiang, M. Zhang, L. Zhou, S. Zheng, A pathogenic role of IL-17 at the early stage of corneal allograft rejection, Transplant immunology 21(3) (2009) 155-161.
H.N. Jacobs, S. Rathod, J. Elisseeff, Intra-Articular Injection of Urinary Bladder Matrix Reduces the Development of Post Traumatic Osteoarthritis, Osteoarthr Cartilage 24 (2016) S432-S432.
N.L. Diny, G.C. Baldeviano, M.V. Talor, J.G. Barin, S. Ong, D. Bedja, A.G. Hays, N.A. Gilotra, I. Coppens, N.R. Rose, Eosinophil-derived IL-4 drives progression of myocarditis to inflammatory dilated cardiomyopathy, Journal of Experimental Medicine 214(4) (2017) 943-957.
Y.P. Goh, N.C. Henderson, J.E. Heredia, A. Red Eagle, J.I. Odegaard, N. Lehwald, K.D. Nguyen, D. Sheppard, L. Mukundan, R.M. Locksley, A. Chawla, Eosinophils secrete IL-4 to facilitate liver regeneration, Proc Natl Acad Sci U S A 110(24) (2013) 9914-9.
Y. Motomura, H. Morita, K. Moro, S. Nakae, D. Artis, T.A. Endo, Y. Kuroki, O. Ohara, S. Koyasu, M. Kubo, Basophil-derived interleukin-4 controls the function of natural helper cells, a member of ILC2s, in lung inflammation, Immunity 40(5) (2014) 758-771.
J.T. Walsh, S. Hendrix, F. Boato, I. Smirnov, J. Zheng, J.R. Lukens, S. Gadani, D. Hechler, G. Gölz, K. Rosenberger, MHCII-independent CD4+ T cells protect injured CNS neurons via IL-4, The Journal of clinical investigation 125(2) (2015) 699-714.
E. Cipolla, A.J. Fisher, H. Gu, E.A. Mickler, M. Agarwal, C.A. Wilke, K.K. Kim, B.B. Moore, R. Vittal, IL-17A deficiency mitigates bleomycin-induced complement activation during lung fibrosis, FASEB journal : official publication of the Federation of American Societies for Experimental Biology 31(12) (2017) 5543-5556.
M.T. Wolf, S. Ganguly, T.L. Wang, C.W. Anderson, K. Sadtler, R. Narain, C. Cherry, A.J. Parrillo, B.V. Park, G. Wang, A biologic scaffold-associated type 2 immune microenvironment inhibits tumor formation and synergizes with checkpoint immunotherapy, Science translational medicine 11(477) (2019) eaat7973.
M. Mohrs, K. Shinkai, K. Mohrs, R.M. Locksley, Analysis of type 2 immunity in vivo with a bicistronic IL-4 reporter, Immunity 15(2) (2001) 303-311.
M.A. Stepp, J.D. Zieske, V. Trinkaus-Randall, B.M. Kyne, S. Pal-Ghosh, G. Tadvalkar, A. Pajoohesh-Ganji, Wounding the cornea to learn how it heals, Experimental eye research 121 (2014) 178-193.

From the foregoing description, it can be apparent that variations and modifications may be made to the invention described herein to adopt it to various usages and conditions. Such embodiments are also within the scope of the following claims.

1. A method of treating an ocular injury, disease or disorder in a subject, comprising:
administering to subject in need thereof an effective amount of a composition comprising one or more biological scaffold materials, wherein the composition increases IL4 production.

2. A method of treating an ocular injury, disease or disorder in a subject, comprising:
administering to subject in need thereof an effective amount of a gel that comprises one or more extracellular matrix materials.

3. The method of claim 1 wherein the subject is in need of cornea repair or reconstruction.

4. The method of claim 1 wherein the subject has suffered an ocular injury, is suffering from an inflammatory ocular disease or disorder, or is an ocular surgery patient.

5. The method of claim 1 wherein the subject is suffering from uveitis, severe conjunctivitis (vernal keratoconjunctivitis), and dry eye syndrome (including, but not limited to, Keratoconjunctivitis sicca and Sjogren’s syndrome), diabetic retinopathy, or autoimmune ocular inflammatory disease.

6. The method of claim 1 wherein the subject is suffering from dry eye disease.

7. The method of claim 1 wherein the composition comprises a biocompatible scaffold that comprises a biocompatible synthetic material, biomaterial or combinations thereof.

8. The method of claim 1 wherein the composition comprises a plurality of particles.

9. The method of claim 8 wherein the particles have a mean particle size of 50 µm or less.

10. The method of claim 1 wherein the composition is administered as a fluid composition to an eye of the subject.

11. The method of claim 1 wherein the composition is administered as drops to an eye of the subject.

12. The method of claim 1 wherein the composition is administered by subconjunctival injection.

13. The method of claim 11 wherein the composition comprises a urinary bladder matrix (UBM) scaffold.

14. The method of claim 1 wherein the biocompatible scaffold further comprises one or more additional therapeutic agents.

15. An eye drop pharmaceutical composition comprising an effective amount of one or more extracellular matrix materials.

16. A kit for treating an ocular injury, disease or disorder, comprising:
(a) a composition that comprises one or more extracellular matrix materials; and
(b) instructions for use of the composition to treat an ocular injury, disease or disorder.
